(19) United States
(12) Patent Application Publication
(10) Pub. No.: US 2016/0319237 A1
(43) Pub. Date: Nov. 3, 2016

(54) MEDIUM COMPOSITION FOR CULTURING MICROALGAE COLONY AND USE THEREOF

(71) Applicant: ADVANCED BIOMASS R&D CENTER, Daejeon (KR)

(72) Inventors: Ji-Won YANG, Daejeon (KR); Nam Kyu KANG, Daejeon (KR); Bongsoo LEE, Daejeon (KR); Sung-Eun SHIN, Daejeon (KR); Eun Kyung KIM, Daejeon (KR); Seung Jib JEON, Daejeon (KR); Min-su KWAK, Daejeon (KR)

(21) Appl. No.: 15/110,762

(22) PCT Filed: Jun. 23, 2014
(86) PCT No.: PCT/KR2014/005520

§ 371 Date: Jul. 11, 2016

(30) Foreign Application Priority Data
Jan. 10, 2014 (KR) 10-2014-0003256

(51) Int. Cl. C12N 1/12

(52) U.S. Cl. CPC C12N 1/12

(57) ABSTRACT

A medium composition for increasing the colony number or colony size of microalgae includes, as an effective component, a conditioned medium derived from culture solution during the exponential phase of microalgae in culture. A solid medium contains the medium composition. A method for increasing the colony number or colony size of microalgae includes spreading and culturing microalgae on the solid medium.

### TECHNICAL FIELD

The present invention relates to a medium composition for culturing microalgae and a use thereof. More specifically, it relates to a medium composition for increasing the colony number or colony size of microalgae characterized by comprising, as an effective component, a conditioned medium derived from culture solution during the exponential phase of microalgae in culture, a solid medium containing the medium composition, and a method for increasing the colony number or colony size of microalgae characterized by spreading and culturing microalgae on the solid medium.

### BACKGROUND ART

To achieve commercialization of a process of producing biodiesel based on microalgae, it is important to enhance the growth rate and lipid content of microalgae. Because it is not economically feasible to have commercialization of existing microalgae, it is important to develop smart microalgae with high lipid content and high growth rate. However, except the microalgae Chlamydomonas reinhardtii which has been studied as a model strain, it is difficult to modify strains of microalgae based on genetic engineering method. One of the reasons is that, unlike bacteria, it takes a long time for the microalgae to form a colony, and there are some species which do not easily form a colony. Thus, if there is difficulty for forming a colony, transformed mutant strains cannot be easily screened.

Nannochloropsis salina is a marine microalga with oleaginous property, and it has very high commercial value. If the strain of Nannochloropsis salina can be successfully modified according to development of a transformation techniques, the possibility of using it as a microalgae having commercially valuable property will be high. Accordingly, it is currently required to develop various techniques of transforming Nannochloropsis salina.

Thus, in the present invention, with regard to a study for obtaining quickly a single colony of Nannochloropsis salina with large size which can be used as a basic tool of the techniques for transformation, an agar plate is prepared by using a conditioned medium on which Nannochloropsis salina has been cultured, and after spreading Nannochloropsis salina on the agar plate, a change in growth of the colony is observed.

Meanwhile, in Korean Patent Publication No. 2011-0062979, “Medium composition for culturing microalgae Nannochloropsis EP1 and use of the microalgae” is described, and in Korean Patent Publication No. 2012-0110295, “Medium composition for culturing algae and method for culturing algae” is described. However, the medium composition for culturing microalgae and use thereof of the present invention are not described.

### DETAILED DESCRIPTION OF THE INVENTION

### Technical Problems to be Solved

The present invention is devised under the circumstances described above, and according to the present invention, an agar plate is prepared by mixing various ratios of a conditioned medium in which a culture solution containing culture of Nannochloropsis salina is used again, and Nannochloropsis salina is smeared and cultured on the agar plate. As a result, it was found that, the colony number or colony size of Nannochloropsis salina is increased on the agar plate in which the conditioned medium is mixed at specific ratio, and the present invention is completed accordingly.

### Technical Means for Solving the Problems

To solve the problems described above, the present invention provides a medium composition for increasing the colony number or colony size of microalgae characterized by comprising, as an effective component, a conditioned medium derived from culture solution during the exponential phase of microalgae in culture.

Also provided by the present invention is a solid medium containing the medium composition.

Also provided by the present invention is a method for increasing the colony number or colony size of microalgae characterized by spreading and culturing microalgae on the solid medium of the present invention.

### Advantageous Effect of the Invention

As a result of culturing the microalgae Nannochloropsis salina, which is difficult to form a colony and requires a long period of time for colony growth, by using a medium containing a conditioned medium, the effect of having increased colony number and increased colony size was confirmed. Because the medium composition of the present invention enables easy screening of transformed strains for study of microalgae and saves cost and time, it is believed that the medium composition can be very effectively used in the industry.

### BRIEF DESCRIPTION OF THE DRAWINGS

FIG. 1 is a drawing illustrating the result of analyzing colony size depending on the ratio of conditioned medium.

FIG. 2 is a photographic image in which colony size of Nannochloropsis salina is compared for a Noble agar plate which has been added with 0% (A) or 30% (B) of the conditioned medium.

FIG. 3 is a result illustrating the result of analyzing a change in colony size depending on the type of agar admixed with the conditioned medium, and time.

### BEST MODE(S) FOR CARRYING OUT THE INVENTION

To achieve the purpose of the present invention, the present invention provides a medium composition for increasing the colony number or colony size of microalgae characterized by comprising, as an effective component, a conditioned medium derived from culture solution during the exponential phase of microalgae in culture.

With regard to the medium composition for increasing the colony number or colony size of microalgae according to one embodiment of the present invention, the conditioned medium is a diluted solution prepared by serial dilution of a medium, which has been obtained by collecting a culture medium at exponential phase based on absorbance measurement during culture of microalgae followed by filtering, with a solvent for dilution at unit of 10% (v/v). Preferably, it may be a diluted solution with dilution ratio of 1/10 to 8/10. More preferably, it may be a diluted solution with dilution ratio of 1/10 to 7/10, but it is not limited thereto. The diluted solution with dilution ratio of 1/10 means a conditioned medium which is obtained by diluting 1 volume of filtered stock medium with 9 volume of a solvent for dilution, and it is used with the same meaning as 10% (v/v).

The medium composition for increasing the colony number of microalgae according to the present invention may contain a conditioned medium as a diluted solution with dilution ratio of 5/10 to 6/10, and it may contain a conditioned medium as a diluted solution with dilution ratio of 6/10, but it is not limited thereto.

Furthermore, the medium composition for increasing the colony size of microalgae according to the present invention may contain a conditioned medium as a diluted solution with dilution ratio of 2/10 to 4/10, and it may contain a conditioned medium as a diluted solution with dilution ratio of 3/10, but it is not limited thereto.

The medium composition for increasing the colony size of microalgae according to the present invention may additionally contain sea salt. It may contain sea salt at minimum or higher concentration at which growth of the microalgae is not affected. Preferably, it may contain sea salt at concentration of 10 to 40 g/l, and more preferably at concentration of 12 to 30 g/l, but not limited thereto.

In the medium composition for increasing the colony number or colony size of microalgae according to other embodiment of the present invention, the medium composition may additionally contain sodium nitrate (NaNO₃), sodium phosphate dihydrate (NaH₂PO₄.2H₂O), f/2 vitamin and f/2 trace metals, and it may preferably contain 350-400 mg/l of sodium nitrate (NaNO₃), 4-8 mg/l of sodium phosphate dihydrate (NaH₂PO₄.2H₂O), 0.1-2 μg/l of vitamin B₁₂ (cyanocobalamin), 0.1-2 μg/l, of biotin, 0.05-0.2 mg/l of thiamine hydrochloride (thiamine.HCl), 3.0-3.5 mg/l of iron chloride hexahydrate (FeCl₃.6H₂O), 4-5 mg/l of Na₂EDTA.2H₂O, 9-11 μg/l of copper sulfate pentahydrate (CuSO₄.5H₂O), 6-7 μg/l of sodium molybdate dihydrate (Na₂MoO₄.2H₂O), 20-30 μg/l of zinc sulfate heptahydrate (ZnSO₄.7H₂O), 5-15 μg/l of cobalt chloride hexahydrate (CoCl₂.6H₂O), and 150-200 μg/l. of manganese chloride tetrahydrate (MnCl₂.4H₂O), but it is not limited thereto.

With regard to the medium composition for increasing the colony number or colony size of microalgae according to other embodiment of the present invention, the microalgae may be algae of Nannochloropsis sp. such as Nannochloropsis salina, Nannochloropsis gaditana, Nannochloropsis granulate, Nannochloropsis limnetica, Nannochloropsis oceanic, or Nannochloropsis oculata, but it is not limited thereto. Preferably, it may be Nannochloropsis salina, but it is not limited thereto.

The present invention also provides a solid medium containing the medium composition for increasing the colony number or colony size of microalgae. As for the method for producing a solid medium by using the medium composition for increasing the colony number or colony size of microalgae, a method well known in the pertinent art can be used. As an example, the conditioned medium is not sterilized but only Bacto and Noble agar are admixed with water and sterilized and then admixed with the conditioned medium at specific ratio to give a solid medium, but it is not limited thereto.

The present invention also provides a method for increasing the colony number or colony size of microalgae characterized by spreading and culturing microalgae on the solid medium of the present invention. Because the medium composition for increasing the colony number or colony size of microalgae is contained in the solid medium of the present invention, when microalgae is smeared by a method like streaking or spreading on the solid medium followed by culturing, the increased colony number or increased colony size of microalgae can be achieved.

Herein below, the present invention is explained in greater detail in view of the Examples. However, it is evident that the following Examples are given only for exemplification of the present invention and by no means the present invention is limited to the following Examples.

### EXAMPLES

### Example 1

### Preparation of Solid Medium Using Conditioned Media

Nannochloropsis salina CCMP 1776 strain purchased from NCMA (National Center for marine algae and microbiota) was inoculated to modified f/2 medium which has been prepared by adding 750 mg/t of nitrate salt to f/2 medium (Guillard, 1975, culture of marine invertebrate animals p. 26-60) containing 30 g/l of sea salt (Sigma, USA), and then cultured while being supplied with 2% carbon dioxide at 0.5 vvm. The inoculation was performed such that, as a reference, the initial microalgae inoculation concentration has absorbance (OD) of 0.2 at 750 nm. After culturing for 1 week, the absorbance was measured and the microalgae culture solution at exponential phase with OD₇₅₀=2.6 was harvested. The harvested culture solution was subjected to a centrifuge treatment at rate of 7,000 rpm for 10 minutes at room temperature. The supernatant was filtered through a 0.22 μm bottle top filter to give a conditioned medium. The obtained conditioned medium was diluted with ultra pure distilled water till to have concentration of 80 to 40% (v/v, conditioned medium/distilled water). For the dilution of 30% or less, the final sea salt concentration was adjusted to 12 g/l by using sea salt and ultra pure distilled water. To the diluted solution, 375 mg/l of sodium nitrate (NaNO₃), 6 mg/l of sodium phosphate dihydrate (NaH₂PO₄.2H₂O), and a suitable amount of f/2 vitamin stock and f/2 trace metal stock were added. For the diluted solution with 0% conditioned medium which has been used as a control, ultra pure distilled water containing the algae dissolved at 30 g/l and added with the above compositions was used. The compositions of the vitamin stock and trace metal stock used in the present invention are shown in the following Table 1.

TABLE 1
Compositions of f/2 vitamin solution and trace metal solution

| | Component | Final concentration | |
|---|---|---|---|
| f/2 Vitamin | Vitamin B₁₂ (cyanocobalamin) | 0.5 | μg/l |
| solution | Biotin | 0.5 | μg/l |
| | Thiamine•HCl | 0.1 | mg/l |
| f/2 Trace metal | FeCl₃•6H₂O | 3.15 | mg/l |
| solution | Na₂EDTA•2H₂O | 4.36 | mg/l |
| | CuSO₄•5H₂O | 9.8 | μg/l |
| | Na₂MoO₄•2H₂O | 6.3 | μg/l |
| | ZnSO₄•7H₂O | 22.0 | μg/l |
| | CoCl₂•6H₂O | 10.0 | μg/l |
| | MnCl₂•4H₂O | 180 | μg/l |

After that, the above solution is admixed with agar to prepare an agar plate medium. For preparing a solid medium, the conditioned medium was not sterilized but only Bacto and Noble agar (BD bioscience, USA) were admixed with water and sterilized, and then admixed with a conditioned medium at specific ratio.

### Example 2

### Analysis of Colony Size and Colony Number of Microalgae

About 50 to 60 colonies of Nannochloropsis salina CCMP 1776 strain were smeared onto the solid medium which has been prepared by the above method, and they were cultured at conditions including 25° C. and light intensity of 120 μmol/m²/s.

2-1. Analysis of Colony Size

Colony size was observed after culturing for 19 days, 24 days, or 30 days. Specifically, by taking an image of every colony from a microscopic photograph, the size was measured, the top 20% and the bottom 20% values were excluded, and the average value of the remaining values was obtained.

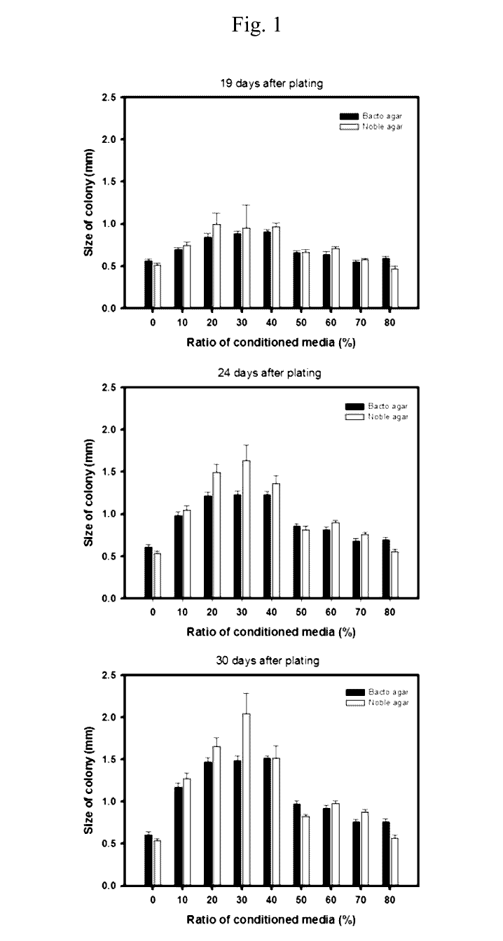

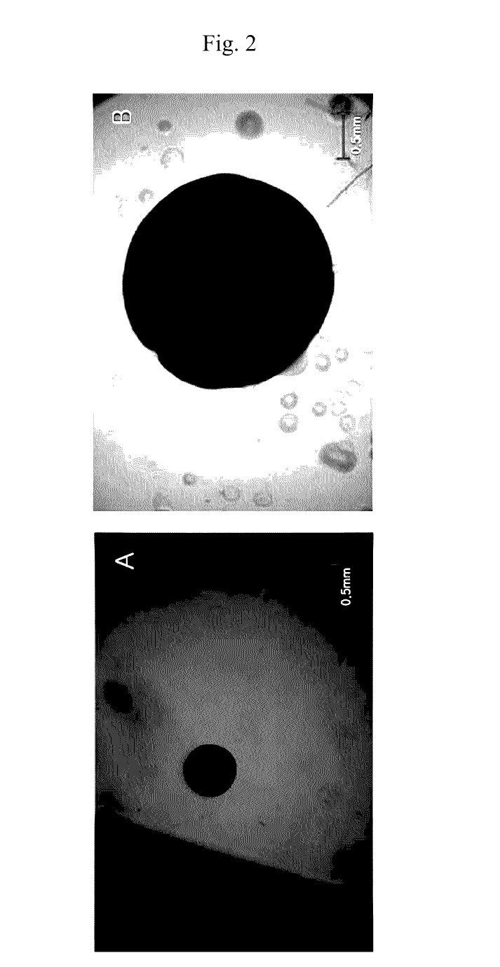

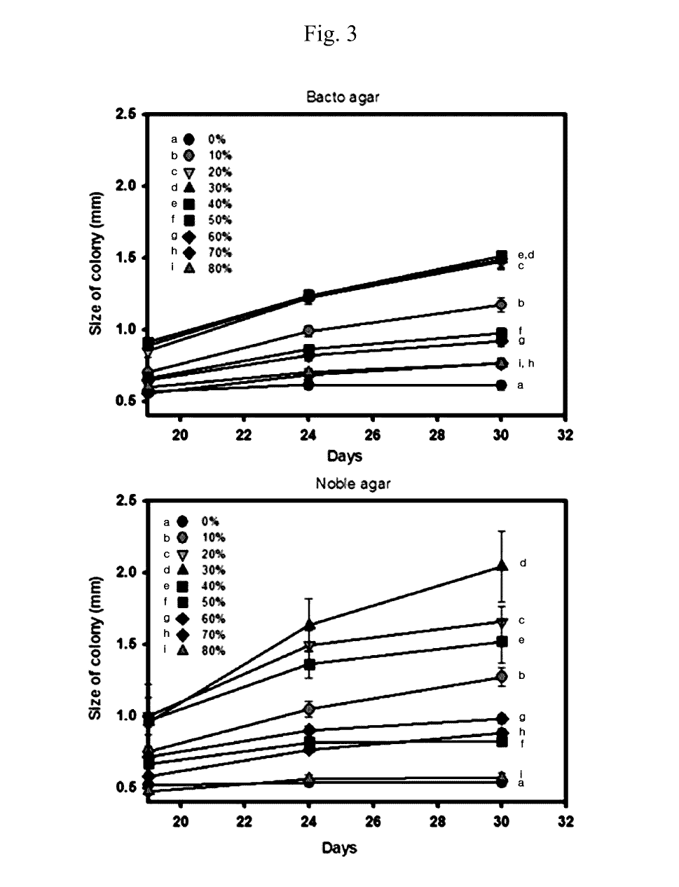

As a result, it was confirmed that the colony size is larger in the medium in which the conditioned medium is used at 20-40% compared to the colony size in a medium with other conditions (FIG. 1). In particular, 30 days after the spreading, the colony in 30% conditioned medium was grown to a size of 2 mm or more, and it is at least 3.8 times larger than the case in which the same agar is used but the conditioned medium is not used (0%) (FIG. 2). It was also confirmed that, during 30 day culture period after the spreading, continuous colony growth has occurred in a medium containing the conditioned medium at 20-40%. Meanwhile, it was found that the colony growth rate is higher in the Noble agar compared to Bacto agar (FIG. 3).

2-2. Analysis of Colony Number

The following Table 2 shows the result of determining colony number of Nannochloropsis salina depending on the ratio of conditioned medium and type of employed agar on day 19 of culture.

TABLE 2
Analysis of colony number depending on ratio
of conditioned medium and type of agar

| | Ratio of conditioned medium | | | | | | | | |
|---|---|---|---|---|---|---|---|---|---|
| | 0% | 10% | 20% | 30% | 40% | 50% | 60% | 70% | 80% |
| Bacto agar | 25 | 54 | 46 | 24 | 43 | 68 | 75 | 51 | 60 |
| Noble | 46 | 37 | 26 | 12 | 32 | 55 | 64 | 60 | 40 |
| agar | | | | | | | | | |

When the conditioned medium is used at 50% or more, 50 or more colonies were shown from all cases except the Noble agar with conditioned medium at 80% ratio. Among them, from the conditioned medium at 60%, 75 colonies were shown from Bacto agar and 64 colonies were shown from Noble agar, indicating the highest growth rate compared to media with other conditions. However, when the conditioned medium is used at 50% or less, 50 or less colonies were observed except the 10% conditioned medium in Bacto agar. In particular, from the 30% conditioned medium from which the largest colony size has been observed, the fewest number of colonies were identified in both agar conditions.

Based on the above result, it was found that 30% conditioned medium in Noble agar is advantageous if fast growth rate and long survival of Nannochloropsis salina colony are needed, while 60% conditioned medium in Bacto agar is advantageous if a large number of colony is needed with increased survival rate of the colony.

1. A medium composition for increasing a colony number or a colony size of microalgae, comprising, as an effective component, a conditioned medium derived from culture solution during an exponential phase of the microalgae in culture.

2. The medium composition according to claim 1, wherein the conditioned medium is a 1/10-8/10 diluted solution of a conditioned medium.

3. The medium composition according to claim 2, wherein the medium composition contains a conditioned medium which is a 5/10-6/10 diluted solution of the conditioned medium.

4. The medium composition according to claim 2, wherein the medium composition contains a conditioned medium which is a 2/10-4/10 diluted solution of the conditioned medium.

5. The medium composition according to claim 4, wherein the medium composition additionally contains sea salt.

6. The medium composition according to claim 1, wherein the medium composition additionally contains sodium nitrate (NaNO₃), sodium phosphate dihydrate (NaH₂PO₄.2H₂O), f/2 vitamin, and f/2 trace metals.

7. The medium composition according to claim 1, wherein the microalgae is a microalgae of Nannochloropsis sp.

8. The medium composition according to claim 7, wherein the microalgae of Nannochloropsis sp. is Nannochloropsis salina.

9. A solid medium containing the medium composition according to claim 1.

10. A method for increasing a colony number or a colony size of microalgae, comprising spreading and culturing microalgae on the solid medium of claim 9.
